(19) United States
(12) Patent Application Publication
(10) Pub. No.: US 2026/0307048 A1
(43) Pub. Date: Oct. 8, 2026

(54) SYSTEM AND METHOD FOR FLUID CAPTURE

(71) Applicants: GE Vernova Infrastructure Technology LLC, Greenville, SC (US); GE VERNOVA TECHNOLOGY GMBH, Baden (CH)

(72) Inventors: Michael Joseph O'BRIEN, Niskayuna, NY (US); Maya CHAABAN, Niskayuna, NY (US); Dana Alexis CAPITANO, Niskayuna, NY (US); Subhadip GOSWAMI, Niskayuna, NY (US); David R. MOORE, Niskayuna, NY (US)

(21) Appl. No.: 19/482,554

(22) PCT Filed: Dec. 6, 2023
(86) PCT No.: PCT/US2023/082739

§ 371 Date: Nov. 7, 2025

Related U.S. Application Data
(63) Continuation of application No. PCT/US2023/021542, filed on May 9, 2023.

(51) Int. Cl. B01J 20/22

(52) U.S. Cl. CPC B01J 20/226; B01J 2220/46

(57) ABSTRACT

In some embodiments, the present disclosure relates to a system. The system includes a substrate and a fluid capture material formed on one or more surfaces of the substrate. The fluid capture material includes a sorbent material that binds one or more fluids, the one or more fluids including water, carbon dioxide, sulfur oxides, alcohols, or a combination thereof. The fluid capture material also includes one or more binder materials, wherein the binder material is optionally at least partially cross-linked. The fluid capture material includes at least one pore.

### RELATED APPLICATIONS

This application claims priority to International Application PCT/US2023/021542, filed on May 9, 2023, the contents of which are hereby incorporated by reference in their entirety.

### BACKGROUND

The subject matter disclosed herein relates to techniques for capturing one or more target fluids. More specifically, the subject matter disclosed herein relates to forming a fluid capture material or coating using a binder and sorbent combination or mixture. The fluid capture material includes at least one pore.

Certain industrial systems may produce a variety of fluids, such as water and carbon dioxide (CO₂), during operation of the industrial systems. In certain instances, the fluids may be vented off as an exhaust gas, or otherwise not utilized. Certain components (e.g., substrates) of the industrial systems may include a coating capable of capturing or extracting the fluids.

### BRIEF DESCRIPTION

Certain embodiments commensurate in scope with the originally filed claims are summarized below. These embodiments are not intended to limit the scope of the present technology, but rather these embodiments are intended only to provide a brief summary of possible forms of the technology. Indeed, the present system and method may encompass a variety of forms that may be similar to or different from the embodiments set forth below.

In one embodiment, the present disclosure relates to a system. The system includes a substrate and a fluid capture material formed on one or more surfaces of the substrate. The fluid capture material includes a sorbent material that binds one or more fluids including water, carbon dioxide, sulfur oxides, alcohols, or a combination thereof. The fluid capture material includes one or more binder materials and the binder material is optionally at least partially cross-linked. The fluid capture material includes at least one pore.

In one embodiment, the present disclosure relates to a method. The method includes providing a sorbent material that binds one or more fluids comprising water, carbon dioxide, sulfur oxides, alcohols, or a combination thereof. The method also includes providing one or more binder materials, wherein the one or more binder materials optionally includes a component capable of forming a cross-linked polymer. Additionally, the method includes optionally providing a cross-linking agent. Additionally, the method includes providing a pore precursor. Further, the method includes generating a sorbent-binder material based on the sorbent material, the one or more binder materials, optionally the cross-linking agent, and the pore precursor. Further still, the method includes applying the sorbent-binder material to a substrate, forming a fluid capture material using the sorbent-binder material applied to the substrate, and removing the pore precursor to form at least one pore in the fluid capture material.

In one embodiment, the present disclosure relates to a system. That system includes a fluid capture material that binds one or more fluids. The fluid capture material includes a sorbent material configured to bind one or more fluids comprising water, carbon dioxide, sulfur oxides, alcohols, or a combination thereof. The fluid capture material also includes a binder material, wherein the binder material is optionally at least partially cross-linked. The fluid capture material also includes at least one pore. Further, the fluid capture material includes an air contactor having one or more surfaces coated with the fluid capture material.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 is a flow diagram of an embodiment of a process for capturing a target fluid using a fluid capture system having one or more substrates, in accordance with the present disclosure;

FIG. 2 is a flow diagram of an embodiment for producing a fluid capture material using a binder and sorbent combination to be used in the fluid capture system of FIG. 1, in accordance with the present disclosure;

FIG. 3 is a cross-sectional view of an embodiment of a substrate coated with the fluid capture material of FIG. 2, in accordance with the present disclosure;

FIG. 4 is a graph depicting a measure of carbon dioxide (CO₂) concentration versus time of a fluid flow directed to a substrate having a fluid capture material, in accordance with the present disclosure:

FIG. 5 is a visual flow diagram illustrating operational aspects of a fluid capture system having one or more substrates coated with a fluid capture material, in accordance with the present disclosure:

FIG. 6 is a graph depicting weight gain versus time of a substrate having a fluid capture material exposed to a fluid flow, in accordance with the present disclosure:

FIG. 7 is a scanning electron microscopy (SEM) image of a cross-section of a film after casting and including wax crystals, in accordance with the present disclosure; and

FIG. 8 is an SEM image of a cross-section of a film after casting and produced using wax crystals, where the wax crystals have been removed, in accordance with the present disclosure.

### DETAILED DESCRIPTION

One or more specific embodiments of the present disclosure will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various examples of the present disclosure, the articles “a,” “an,” “the,” and “said” are intended to mean that there are one or more of the elements. The terms “comprising,” “including,” and “having” are intended to be inclusive and mean that there may be additional elements other than the listed elements. Additionally, it should be understood that references to “one example” or “an example” of the present disclosure are not intended to be interpreted as excluding the existence of additional examples that also incorporate the recited features.

In the present context, the term “about” or “approximately” is intended to mean that the values indicated are not exact and that the actual value may vary from those indicated in a manner that does not materially alter the operation concerned. For example, the term “about” or “approximately” as used herein is intended to convey a suitable value that is within a particular manufacturing or operating tolerance (e.g., ±10%, ±5%, ±1%, ±0.5%), as would be understood by one skilled in the art.

As generally discussed herein, certain systems (e.g., gas turbines) that produce one or more fluids (e.g., water and/or CO₂) may include one or more substrates having a surface coating that binds the one or more fluids, thereby extracting or capturing the one or more fluids from a source fluid (e.g., an exhaust gas flow, an ambient air flow, and the like). For example, the systems may include combustion systems that utilize a fuel source (e.g., fossil fuels). Accordingly, one or more substrates of the combustion systems may include a surface coating capable of extracting carbon dioxide. As another non-limiting example, the systems may include a water capture system that generally includes a surface coating capable of extracting water from ambient air. In certain embodiments, it may be desirable to capture at least a portion of the fluids, such as to address guidance by certain entities (e.g., government regulation) and/or to utilize the one or more fluids rather than otherwise not capturing the fluids, such as by venting or otherwise releasing the fluids into a surrounding airspace or other proximate environment.

The present disclosure is directed to techniques for improving the efficiency of capturing or extracting certain fluids from a fluid flow by forming a fluid capture material or fluid capture coating using a sorbent material (e.g., sorbent component) and a binder material that is optionally capable of cross-linking, and optionally cross-linking the binder material (e.g., using a cross-linking agent). As described in more detail herein, the sorbent material generally includes materials capable of binding certain fluids, such as carbon dioxide (CO₂), water (H₂O), oxygen (O₂), or other gas molecules that may form as result of a decomposition reaction (e.g., combustion). For example, the sorbent materials may include metal organic frameworks (MOFs) and/or covalent-organic frameworks (COFs). In some embodiments, the sorbent materials may include polymeric resins, silicas, zeolites, and the other materials capable of capturing fluids as discussed herein. The binder material may include one or more materials that may block, reduce, or mitigate decomposition or dissolution (i.e., improve the stability) of the sorbent materials. As described in further detail herein, it is presently recognized that forming a fluid capture material using a sorbent material and an optionally cross-linked binder material may provide an improved capacity for binding fluids (e.g., in a reversible or irreversible manner) as compared to conventional fluid binding materials or coatings.

Generally, the sorbent may be any suitable sorbent known in the art that facilitates the sorbent described herein. In some embodiments, the sorbent is selected from the group consisting of coordination framework compounds, metal-organic framework (MOF) compounds, porous coordination polymers (PCPs), covalent organic framework (COF) compounds, zeolitic imidazolate framework (ZIF) compounds, crystalline porous materials, crystalline open frameworks, reticular chemistry, silica particles, zeolites, silico-alumino-phosphates (SAPOs), alumino-phosphates (AlPOs), polyaromatic frameworks (PAFs), activated carbons, molecular organic solids, and combinations thereof.

As used herein, MOF compounds are a class of compounds including metal ions or clusters coordinated to organic ligands to form one-, two-, or three-dimensional structures. The metal ions or clusters act as joints and are bound by multidirectional organic ligands, which act as linkers in a network structure. MOF compounds have a modular nature that allows for synthetic tunability, which affords fine chemical and structural control. Properties such as porosity, stability, particle morphology, and conductivity can be tailored for specific applications.

In many embodiments, the sorbent is a MOF compound including a MOF metal or metal-containing cluster and a MOF linker.

In some embodiments, the MOF metal may be any suitable MOF metal known in the art that facilitates the sorbent described herein. In other embodiments, the MOF metal is a metal selected from the group consisting of alkali metals, alkaline earth metals, transition metals, Ca, Mn, Cr, Fe, Co, Ni, Cu, Zn, ions thereof, hydrates thereof, salts thereof, halides thereof, fluorides thereof, chlorides thereof, bromides thereof, iodides thereof nitrates thereof, acetates thereof, sulfates thereof, phosphates thereof, carbonates thereof, oxides thereof, formates thereof, carboxylates thereof, and combinations thereof. In some embodiments, the MOF metal includes Mg.

In some embodiments, the MOF metal-containing cluster may be any suitable MOF metal-containing cluster known in the art that facilitates the sorbent described herein. In some embodiments, the MOF metal-containing cluster includes an MOF metal node and a linker strut, with the MOF metal and the linker each defined as described herein. In other embodiments, the MOF metal-containing cluster includes an MOF metal-oxy cluster.

In some embodiments, the MOF linker may be any suitable MOF linker known in the art that facilitates the sorbent described herein. Generally, the geometry and connectivity of a linker contribute to the structure of the resulting MOF compound. Adjustments of linker geometry, length, ratio, and functional-group can tune the size, shape, and internal surface property of a MOF compound for a targeted application.

In at least some embodiments, the MOF linker is a linker selected from the group consisting of polytopic linkers, ditopic linkers, tritopic linkers, tetratopic linkers, pentatopic linkers, hexatopic linkers, heptatopic linkers, octatopic linkers, mixed linkers, desymmetrized linker, metallo linkers, N-heterocyclic linkers, and combinations thereof.

In at least some embodiments, the MOF linker is a linker selected from the group consisting of polytopic linkers, 4,4′-dihydroxy-[1,1′-biphenyl]-3,3′-dicarboxylic acid (H₄dobpdc), 4,4′-dioxidobiphenyl-3,3′-dicarboxylate (dobpdc⁴⁻), 4,4″-dioxido-[1,1′:4′,1″-terphenyl]-3,3″-dicarboxylate (dotpdc⁴⁻), 2,5-dioxidobenzene-1,4-dicarboxylate (dobdc⁴⁻), 4,6-Dihydroxyisophthalic acid (m-dobdc⁴⁻), 3,3′-dioxido-biphenyl-4,4′-dicarboxylate (para-carboxylate-dobpdc⁴⁻), 4,4′-[oxalylbis(imino)]bis(2-hydroxybenzoic acid) (H₄ODA), 4,4′-[1,4-phenylenebis-(carbonylimino)]bis(2-hydroxybenzoic acid) (H₄TDA), 4,4′-Dihydroxyazobenzene-3,3′-dicarboxylic acid (H₄OSA), protonated, partially and fully deprotonated forms thereof, and combinations thereof. As another example, in at least some embodiments, the MOF linker is a linker selected from the group consisting of dicarboxylates (e.g., terephthalic acid), tricarboxylates (e.g., 1,3,5-benzentricarboxylic acid), azolates, tetrazolates, and combinations thereof.

As another example, in at least some embodiments, the MOF linker is a dicarboxylic acid linker selected from the group consisting of 1,4-butanedicarboxylic acid, 4-oxopyran-2,6-dicarboxylic acid, 1,6-hexanedicarboxylic acid, decanedicarboxylic acid, 1,8-heptadecanedicarboxylic acid, 1,9-heptadecanedicarboxylic acid, heptadecanedicarboxylic acid, acetylenedicarboxylic acid, 1,2-benzenedicarboxylic acid, 2,3-pyridinedicarboxylic acid, pyridine-2,3-dicarboxylic acid, 1,3-butadiene-1,4-dicarboxylic acid, 1,4-benzenedicarboxylic acid, p-benzenedicarboxylic acid, imidazole-2,4-dicarboxylic acid, 2-methylquinoline-3,4-dicarboxylic acid, quinoline-2,4-dicarboxylic acid, quinoxaline-2,3-dicarboxylic acid, 6-chloroquinoxaline-2,3-dicarboxylic acid, 4,4′-diaminophenylmethane-3,3′-dicarboxylic acid, quinoline-3,4-dicarboxylic acid, 7-chloro-4-hydroxyquinoline-2,8-dicarboxylic acid, diimidedicarboxylic acid, pyridine-2,6-dicarboxylic acid, 2-methylimidazole-4,5-dicarboxylic acid, thiophene-3,4-dicarboxylic acid, 2-isopropylimidazole-4,5-dicarboxylic acid, tetrahydropyran-4,4-dicarboxylic acid, perylene-3,9-dicarboxylic acid, perylenedicarboxylic acid, Pluriol E 200-dicarboxylic acid. 3,6-dioxaoctanedicarboxylic acid, 3,5-cyclohexadiene-1,2-dicarboxylic acid, octanedicarboxylic acid, pentane-3,3-carboxylic acid, 4,4′-diamino-1,1′-diphenyl-3,3′-dicarboxylic acid, 4,4′-diaminodiphenyl-3,3′-dicarboxylic acid, benzidine-3,3′-dicarboxylic acid, 1,4-bis-(phenylamino)benzene-2,5-dicarboxylic acid, 1,1′-dinaphthyl-8,8′-dicarboxylic acid, 7-chloro-8-methylquinoline-2,3-dicarboxylic acid, 1-anilinoanthraquinone-2,4′-dicarboxylic acid, polytetrahydrofuran-250-dicarboxylic acid, 1,4-bis(carboxymethyl)piperazine-2,3-dicarboxylic acid, 7-chloroquinoline-3,8-dicarboxylic acid, 1-(4-carboxy)phenyl-3-(4-chloro) phenylpyrazoline-4,5-dicarboxylic acid, 1,4,5,6,7,7,-hexachloro-5-norbornene-2,3-dicarboxylic acid, phenylindanedicarboxylic acid, 1,3-dibenzyl-2-oxoimidazolidine-4,5-dicarboxylic acid. 1,4-cyclohexanedicarboxylic acid, naphthalene-1,8-dicarboxylic acid, 2-benzoylbenzene-1,3-dicarboxylic acid, 1,3-dibenzyl-2-oxoimidazolidine-4,5-cisdicarboxylic acid, 2,2′-biquinoline-4,4′-dicarboxylic acid, pyridine-3,4-dicarboxylic acid, 3,6,9-trioxaundecanedicarboxylic acid, o-hydroxybenzophenonedicarboxylic acid, Pluriol E 300-dicarboxylic acid, Pluriol E 400-dicarboxylic acid, Pluriol E 600-dicarboxylic acid, pyrazole-3,4-dicarboxylic acid. 2,3-pyrazinedicarboxylic acid, 5,6-dimethyl-2,3-pyrazinedicarboxylic acid, 4,4′-diaminodiphenyletherdiimidedicarboxylic acid, 4,4′-diaminodiphenylmethanediimidedicarboxylic acid, 4,4′-diaminodiphenylsulfonediimidedicarboxylic acid, 2,6-naphthalenedicarboxylic acid, 1,3-adamantanedicarboxylic acid, 1,8-naphthalenedicarboxylic acid, 2,3-naphthalenedicarboxylic acid, 8-methoxy-2,3-naphthalenedicarboxylic acid, 8-nitro-2,3-naphthalenedicarboxylic acid, 8-sulfo-2,3-naphthalenedicarboxylic acid, anthracene-2,3-dicarboxylic acid, 2′-3′-diphenyl-p-terphenyl-4,4″-dicarboxylic acid, diphenylether-4,4′-dicarboxylic acid, imidazole-4,5-dicarboxylic acid, 4(1H)-oxothiochromene-2,8-dicarboxylic acid, 5-t-butyl-1,3-benzenedicarboxylic acid, 7,8-quinolinedicarboxylic acid, 4,5-imidazoledicarboxylic acid, 4-cyclohexene-1,2-dicarboxylic acid, hexatriacontanedicarboxylic acid, tetradecanedicarboxylic acid, 1,7-heptanedicarboxylic acid, 5-hydroxy-1,3-benzenedicarboxylic acid, pyrazine-2,3-dicarboxylic acid, furan-2,5-dicarboxylic acid, 1-nonene-6,9-dicarboxylic acid, eicosenedicarboxylic acid, 4,4′-dihydroxydiphenylmethane-3,3′-dicarboxylic acid, 1-amino-4-methyl-9,10-dioxo-9,10-dihydroanthracene-2,3-dicarboxylic acid, 2,5-pyridinedicarboxylic acid, cyclohexene-2,3-dicarboxylic acid, 2,9-dichlorofluorubin-4,11-dicarboxylic acid, 7-chloro-3-methylquinoline-6,8-dicarboxylic acid, 2,4-dichlorobenzophenone-2′,5′-dicarboxylic acid, 1,3-benzenedicarboxylic acid, 2,6-pyridinedicarboxylic acid, 1-methylpyrrole-3,4-dicarboxylic acid, 1-benzyl-1H-pyrrole-3,4-dicarboxylic acid, anthraquinone-1,5-dicarboxylic acid, 3,5-pyrazoledicarboxylic acid, 2-nitrobenzene-1,4-dicarboxylic acid, heptane-1,7-dicarboxylic acid, cyclobutane-1,1-dicarboxylic acid. 1,14-tetradecanedicarboxylic acid, 5,6-dehydronorbomane-2,3-dicarboxylic acid, 5-ethyl-2,3-pyridinedicarboxylic acid, and combinations thereof.

As another example, in at least some embodiments, the MOF linker is a tricarboxylic acid linker selected from the group consisting of 2-hydroxy-1,2,3-propanetricarboxylic acid, 7-chloro-2,3,8-quinolinetricarboxylic acid, 1,2,4-benzenetricarboxylic acid, 1,2,4-butanetricarboxylic acid, 2-phosphono-1,2,4-butanetricarboxylic acid, 1,3,5-benzenetricarboxylic acid, 1-hydroxy-1,2,3-propanetricarboxylic acid. 4,5-dihydro-4,5-dioxo-1H-pyrrolo[2,3-F]quinoline-2,7,9-tricarboxylic acid, 5-acetyl-3-amino-6-methylbenzene-1,2,4-tricarboxylic acid, 3-amino-5-benzoyl-6-methylbenzene-1,2,4-tricarboxylic acid, 1,2,3-propanetricarboxylic acid, aurinetricarboxylic acid, and combinations thereof.

As another example, in at least some embodiments, the MOF linker is a tetracarboxylic acid linker selected from the group consisting of 1,1-dioxide-perylo[1,12-BCD] thiophene-3,4,9,10-tetracarboxylic acid, perylenetetracarboxylic acids, perylene-3,4,9,10-tetracarboxylic acid, perylene-1,12-sulfone-3,4,9,10-tetracarboxylic acid, butanetetracarboxylic acids, 1,2,3,4-butanetetracarboxylic acid, meso-1,2,3,4-butanetetracarboxylic acid, decane-2,4,6,8-tetracarboxylic acid, 1,4,7,10,13,16-hexaoxacyclooctadecane-2,3,11,12-tetracarboxylic acid, 1,2,4,5-benzenetetracarboxylic acid, 1,2,11,12-dodecanetetracarboxylic acid. 1,2,5,6-hexanetetracarboxylic acid, 1,2,7,8-octanetetracarboxylic acid. 1,4,5,8-naphthalenetetracarboxylic acid, 1,2,9,10-decanetetracarboxylic acid, benzophenonetetracarboxylic acid, 3,3′,4,4′-benzophenonetetracarboxylic acid, tetrahydrofurantetracarboxylic acid, cyclopentanetetracarboxylic acids, cyclopentane-1,2,3,4-tetracarboxylic acid, and combinations thereof.

In the exemplary embodiment, the MOF linker is 4,4′-dihydroxy-[1,1′-biphenyl]-3,3′-dicarboxylic acid (H₄dobpdc) and/or 4,4′-dioxidobiphenyl-3,3′-dicarboxylate (dobpdc⁴⁻). In some embodiments, dobpdc includes 4,4′-dihydroxy-[1,1′-biphenyl]-3,3′-dicarboxylic acid, its mono-carboxylate form, its di-carboxylate form, its mono-phenoxide form, its di-phenoxide form, and combinations thereof.

In some embodiments, the MOF linker is one or more of the following linkers:

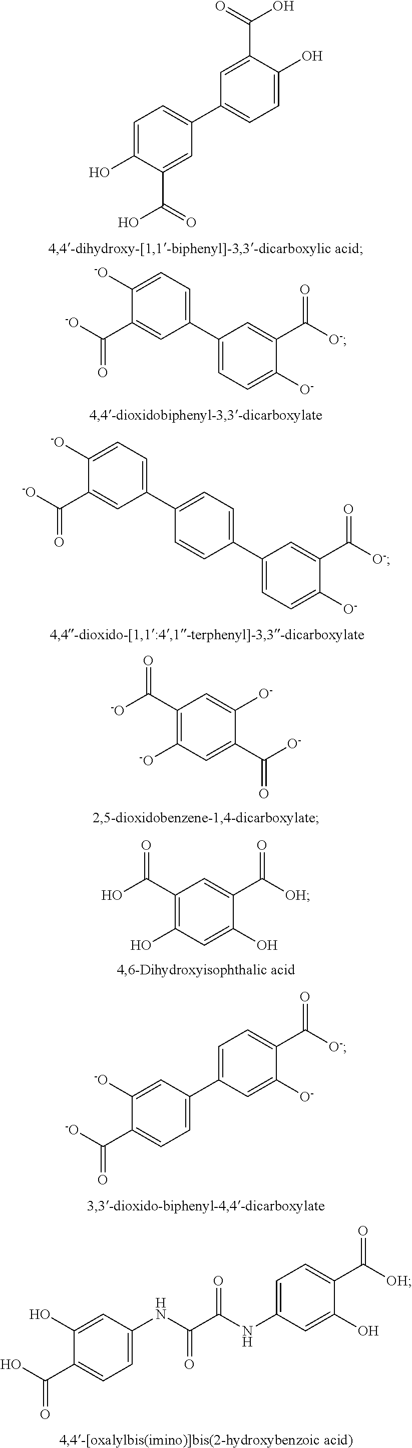

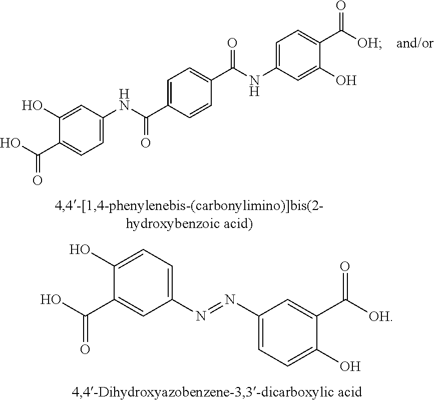 [SMILES: O=C(Nc1ccc(C(=O)O)c(O)c1)c1ccc(C(=O)Nc2ccc(C(=O)O)c(O)c2)cc1 ; O=C(O)c1cc(/N=N/c2ccc(O)c(C(=O)O)c2)ccc1O]

In some embodiments, the MOF compound is a MOF compound of the MOF-74 family. In some embodiments, the MOF compound is a MOF compound of the MOF-274 family. In some embodiments, the MOF compound is a MOF compound of the MOF-303 family. In some embodiments, the MOF compound is Mg₂(dobpdc).

The disclosed fluid capture material or coating may be formed by cross-linking a binder material that is capable of forming a cross-linked polymer. At least in some instances, the disclosed fluid capture material may include a portion (e.g., a mass percentage) that is a cross-linked polymer (e.g., a cross-linked binder material). By way of example, such a portion may be less than 20% by mass, between 1% and 15% by mass, between 5% and 10% by mass, or less than 10% by mass of the total mass of the fluid capture material. In general, the cross-linked polymer may be formed using thermal techniques, radiation techniques (e.g., illumination with ultraviolet (UV) light) and/or chemical techniques (e.g., using cross-linking agents via radical polymerization or condensation reactions). In embodiments where a cross-linking agent is used, the fluid capture material may also include the cross-linking agent. That is, the cross-linking agent may be present in the fluid capture material. It is presently recognized that a fluid capture material that includes a cross-linked polymer (e.g., the fluid capture material is formed using the cross-linked polymer) may result in a fluid capture material that has a relatively higher amount of the sorbent (e.g., a relatively lower amount of the binder material (e.g., less than 15%, less than 12% by mass, less than 10% by mass, less than 8% by mass, less than 5% by mass) as compared to a fluid capture material formed using a binder or polymer that is not cross-linked and/or is incapable of cross-linking. Accordingly, increasing the amount of the sorbent material improves the fluid binding capacity of the fluid capture material by having a higher amount of the sorbent material in the fluid capture material. Furthermore, by forming the fluid capture material with a cross-linked polymer, the disclosed fluid capture material may have improved adherence or binding to a substrate (e.g., a metal substrate, polymeric substrate (e.g., a glass-filled nylon), polymer composite substrate, and the like) and stability or resistance to dissolution.

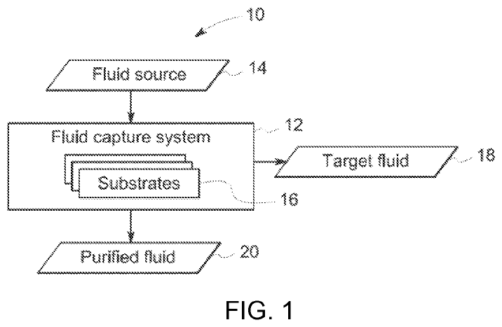

With this in mind, FIG. 1 is a flow diagram of an embodiment of a process 10 for capturing or extracting a fluid from a fluid flow. As illustrated, a fluid capture system 12 receives fluid from a fluid source 14. In general, the fluid source 14 may include an exhaust fluid flow (e.g., an exhaust gas flow) and/or ambient air. As described herein, the fluid source 14 may include one or more target fluids (e.g., one or more target gases) that may be desirable to capture or otherwise extract or isolate from the exhaust fluid flow. For example, it may be desirable to capture certain products of combustion. That is, in certain instances, it may be desirable to capture CO₂ to reduce an amount of CO₂ emissions into the environment (e.g., in accordance with certain regulations). Additionally or alternatively, it may be desirable to capture H₂O to reduce a moisture content of an air flow. As another non-limiting example, it may be advantageous to capture certain sulfur oxides (SOₓ) produced from an exhaust gas. In any case, the fluid capture system 12 generally receives the fluid from the fluid source 14 and one or more substrates 16 of the fluid capture system 12 extract one or more target fluids 18 from the fluid of the fluid source 14, thereby generating a purified gas flow 20.

In certain embodiments, the fluid capture system 12 may be provided as part of a gas turbine system, a chemical production system, or other systems that produce a fluid flow (e.g., a gas flow, an exhaust gas flow) having gas molecules that may be desirable to capture. As illustrated, the fluid capture system 12 may include one or more substrates 16. As described herein, the substrates 16 may include a coating formed of a semi-permeable material or materials (e.g., capable of letting certain gases permeate through the substrates) that is capable of binding certain fluids (i.e., the target fluid 18 or gas). For example, the coating may be a fluid capture material formed using a sorbent material and a binder material that is capable of forming a cross-linked polymer.

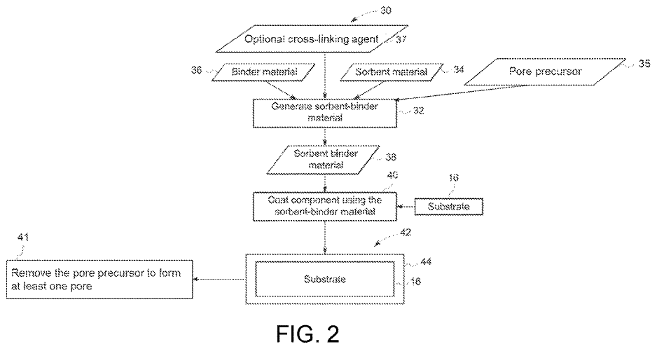

As described herein, the fluid capture material may improve the amount of target fluid 18 extracted from the fluid source 14 and/or may have improved stability as compared to certain coatings used to extract fluids from a fluid source 14. To illustrate this, FIG. 2 is a flow diagram of an embodiment of a process 30 for producing an air contact with a fluid capture material.

To start the process 30, at block 32, a sorbent material 34, a binder material 36, optionally a cross-linking agent 37, and a pore precursor 35 are used to generate a sorbent-binder material 38. In general, using the sorbent material 34, the binder material 36, optionally the cross-linking agent 37, and the pore precursor 35 may include forming a mixture, such as a solution or slurry including the sorbent material 34 and the binder material 36 and optionally the pore precursor 35 in a suitable solvent that is capable of dissolving at least a portion of the sorbent material and/or the binder material. Examples of such solvents include, but are not limited to, toluene, ethyl acetate, ethanol, 2-(2-butoxyethoxy) ethyl acetate, water, isopropanol, methyl ethyl ketone, or any combination thereof (i.e., for miscible solvents). As discussed herein, the cross-linking agent 37 may include certain chemical cross-linking agents. As such, the cross-linking agents 37 may also be added to the mixture of the sorbent material 34 and the binder material 36. In some embodiments, at least one of the cross-linking agent 37 and the pore precursor 35 may be added after forming the mixture of the sorbent material 34 and the binder material 36. For example, in an embodiment where the binder material 36 is a polymeric material, at least one of the cross-linking agent 37 and the pore precursor 35 may be added after a time period corresponding to a suitable degree of polymerization of the binder material 36 (e.g., after initiation of polymerization of the binder material 36). However, in certain embodiments, at least one of the cross-linking agent 37 and the pore precursor 35 may be added prior to polymerization of the binder material 36 being initiated.

The sorbent material 34 is generally a material capable of adsorbing fluids such as water and/or CO₂. In some embodiments, the sorbent material 34 may include metal-organic frameworks (MOFs) and/or covalent-organic frameworks (COFs). For example, the sorbent material may include MOFs such as iron-based MOFs, zirconium-based MOFs (e.g., MOF-808, such as MOF-808-Gly), aluminum-based MOFs (e.g., MOF-303, MIL-160), zeolitic imidazolate frameworks (ZIFs), amine-containing MOFs, other MOFs, amine-containing COFs, ZIFs, silicas, and the like, that are capable of adsorbing fluids as described herein. In some embodiments, the sorbent material 34 may include polymeric resins, silicas, zeolites, or a combination thereof.

The binder material 36 may include one or more oligomeric or polymer materials, monomeric or oligomeric material capable of polymerizing, or a combination thereof. At least in some instances, the binder material 36 may improve the affinity of the sorbent material 34 for binding a certain gas or gases and/or improve the stability (e.g., thermostability) of the sorbent material 34. In some embodiments, the binder material 36 may include materials that form polymers having a thermostability about 200° C. In some embodiments, the binder material 36 may include silicon-containing polymers or binders (e.g., siloxanes or silanes, such as aminopropylsilsesquioxane, aminoethylaminopropylsilsesquioxane, alkyoxysilane), vinyl polymers (e.g., polyvinyl esters, such as polyvinyl acetate; polyvinyl alcohol) and copolymers thereof like polyvinyl butyral. In some embodiments, the binder material 36 may include polysaccharides (e.g., ethyl cellulose, starch, and alkyl cellulose), nitrogen-containing polymers (e.g., polyethyleneimine (PEI)). In some embodiments, the binder material 36 may include combinations of the previously described polymers (i.e., 2, 3, 4, or more than 4 of the polymers). For example, the binder material 36 may be a “hybrid binder mixture.” As referred to herein, a “hybrid binder mixture” may include mixtures or blends of different types of binder materials, such as a mixture of organic polymers and silsesquioxane binders, or other combinations of binder materials described herein. At least in some instances, the binder material 36 may be selected to enhance the adsorption of the target fluid onto a coating (e.g., the fluid capture material) produced using the sorbent material 34. For example, in an embodiment where PEI is used as a binder material, the PEI may include PEI-low (e.g., M_{W} between approximately 20,000 g/mol and 25,000 g/mol, and Mₙ between approximately 8,000 g/mol to 12.000 g/mol) or PEI-high (e.g., M_{W} between approximately 70,000 g/mol and 80,000 g/mol, and Mₙ between approximately 55,000 g/mol to 65,000 g/mol).

As described herein, the binder material 36 may be a polymer material that is capable of cross-linking. That is, it is presently recognized that forming a fluid capture material where at least a portion of a polymer portion of the sorbent-binder material 38 is a cross-linked polymer may reduce the likelihood of decomposition and/or dissolution of the sorbent material 34. Further, using a cross-linked polymer may enable the fluid capture material to have a relatively higher amount of the sorbent material that binds to the target fluid 18, and thus may have a higher fluid binding capacity as compared to a coating formed without cross-linked polymers. Put differently, conventional techniques of combining of sorbent material 34 and a binder material 36 may result in a fluid binding material that has a relatively lower fluid binding capacity as compared to the sorbent material (e.g., due to a dilution effect or knockdown effect). It is presently recognized that cross-linking the binder material 36 may generate a fluid capture coating or fluid capture material that has a relatively higher binding capacity as compared to not cross-linking the binder material 36. Further, the binding capacity of the disclosed fluid capture coating or material (i.e., including a cross-linked binder material) may have a binding capacity that is approximately equal to the binding capacity of the sorbent material 34 by itself (e.g., the sorbent material 34 in powder form).

In one embodiment, the binder material 36 includes materials that are capable of self-cross-linking. For example, the binder material 36 may include silanol (SiOH) functional groups and/or alkoxysilane (SiOR) functional groups. It should be noted that binder materials 36 that include such functional groups may undergo intermolecular condensation reactions that cause the binder materials 36 to crosslink upon heating. For example, it is presently recognized that an amine-containing component (e.g., an amine-containing MOF) may cause certain binder materials 36 (e.g., epoxy resins) to cross-link. As another non-limiting example, an amine-containing component may cross-link certain Si—O polymeric structures, such as silsesquioxane, thereby forming a cross-linked Si—O polymeric structure (e.g., amine-impregnated silica).

In one embodiment, the binder material comprises a polyvinyl alcohol polymer. Suitable polyvinyl alcohol polymers include, without limitation polyvinyl alcohol homopolymers, and polyvinyl alcohol copolymers. In one embodiment, the binder polymer composition comprises a polyvinyl alcohol-polyvinyl amine copolymer (PVA-PVAm) comprising a first crosslinkable functional group and a second crosslinkable functional group. Although derivatives of polyvinyl alcohol are suitable for the practice of the present invention, other polymeric materials may be used in the binder polymer composition, including without limitation, polyacrylates, polymethacrylates, polyhydroxyethyl methacrylates, functionalized polyarylenes containing amine, carboxylic acid, amide, hydroxyl moieties, and the like. In one embodiment, the binder polymer composition used in the preparation of the fluid capture material comprises at least one polymer having a number average molecular weight greater than about 2500 Daltons. In another embodiment, the binder polymer composition used in the preparation of the fluid capture material comprises at least one polymer having a number average molecular weight in a range of from greater than 2500 Daltons to about 500,000 Daltons. In yet another embodiment, the binder polymer composition used in the preparation of the fluid capture material comprises at least one hydrophilic polymer having a number average molecular weight in a range of from about 75,000 Daltons to about 250,000 Daltons. Number average molecular weights may be determined by a variety of techniques known to those of ordinary skill in the art including ¹H-NMR spectroscopy and gel permeation chromatography (GPC).

As described above, the binder material 36 may include a mixture of polymer materials that are capable of cross-linking. For example, the binder material 36 may include a mixture of polyvinyl alcohol (PVA) and polyacrylic acid (PAA). For example, the mixture may include 10% by weight of PVA and 90% by weight of PAA, 30% by weight of PVA and 70% by weight of PAA, 50% by weight of PVA and 50% by weight of PAA, 70% by weight of PVA and 30% by weight of PAA, or 90% by weight of PVA and 10% by weight of PAA.

In some embodiments, the binder material 36 may be dissolved in a solvent to a particular viscosity. For example, in an embodiment where the binder material 36 includes ethyl cellulose, the binder material 36 may include a 7-15 cP solution in 6% toluene in ethanol solution. The resulting slurry may include 30% solids, 11% binder, when dissolved in a 1:1 toluene 2-(2-butoxyethoxy) ethyl acetate solvent. As another non-limiting example, in an embodiment where the binder material 36 includes ethyl cellulose, the binder material 36 may include an approximately 300 cP solution in 5% toluene in ethanol solution.

In general, the amount of cross-linking agent 37 may be less than the amount of binder material 36. In some embodiments, the ratio of cross-linking agent 37 added to the binder material 36 to form the sorbent binder composite 38 may be less than approximately 1/3, less than approximately 1/4, less than approximately 1/5, or less than approximately 1/6. For example, the sorbent binder composite 38 may be formed by combining a 10% by mass solution of a binder material 36 and a 2% by mass solution of a cross-linking agent 37 (i.e., the ratio of the cross-linking agent 37 to the binder material 36 is 1/5).

It should be noted that, at least in some instances, the cross-linking agent 37 may also be a binder material 36. That is, the cross-linking agent 37 may be a polymer that is capable of cross-linking. For example, PAA may be used as a cross-linking agent for PVA.

As described herein, the cross-linking agent 37 cross-links the binder material 36. In some embodiments, the degree of cross-linking (i.e., cross-linking density, which refers to the density of chains or segments that connect two parts of a polymer network, rather than the density of cross-link junctures) may be greater than 10%, greater than 20%, greater than 30%, greater than 40%, greater than 50%, greater than 60%, greater than 70%, greater than 80%, or greater than 90%.

With respect to the sorbent-binder material 38 (e.g., the sorbent-binder composite), the amount of binder material 36 and sorbent material 34 may be such that the sorbent-binder material 38 includes greater than 50%, of the sorbent material, greater than 60% of the sorbent material, greater than 70% of the sorbent material, greater than 80% of the sorbent material 34, greater than 85% of the sorbent material 34, or greater than 90% of the sorbent material 34.

A wide variety of cross-linking agents can be used to react with the binder, and these cross-linking agents may be monomers, oligomers, or polymers, or a combination of the foregoing. In some embodiments, the cross-linking agent 37 may include chemical cross-linking agents such as epoxies, anhydrides, and the like. In some embodiments, the cross-linking agent 37 may include one or more materials such as nanoparticles, micron-sized particles, or larger sized particles, or molecular precursors that can form particles. For example, the cross-linking agent may include silica particles, such as colloidal silica: or tetraalkoxysilane that can form silica particles. In some embodiments, the cross-linking agent 37 may include particles having different size distributions. That is, the cross-linking agent 37 may include particles of a first size distribution and a second size distribution. For example, the cross-linking agent 37 may have a micron-size distribution. In some embodiments, the cross-linking agent 37 may have a nano-size distribution and a micron-size distribution (i.e., a bimodal size distribution). At least in some instances, a bimodal size distribution may improve wear resistance. In an embodiment where the cross-linking agent 37 includes particles having different size distributions, the mixture of the particles may vary. For example, the mixture may include 10%, 20%. 30%, 40%, 50%, 60%. 70%, and the like, by weight, of the nano-sized particles and 90%, 80%, 70%, 60%, 50%, 40%, 30%, and the like, by weight, of the micron-sized particles. In an embodiment where the cross-linking agent 37 includes particles (e.g., micron-sized particles, nanoparticles, or larger particles), the particles may have a distribution of shapes. For example, the cross-linking agent 37 may include micron-sized particles that are 10%, 20%, 30%, 40%, 50%, 60%, 70%, 80% 90%, or 95% spherical. At least in some instances, the combination of particle shape (e.g., spherical) and different size distributions may improve the properties of the resulting fluid capture material discussed herein.

In some embodiments, the cross-linking agent 37 comprises a functional group susceptible to the formation of a free radical resulting from exposure to high energy irradiation (e.g., ultraviolet light or electron beam) and/or heat. Those of ordinary skill in the art will appreciate that the structure of a free radical is understood to determine its reactivity and that the structures of the cross-linking agent may be selected to provide for a higher or lower level of chemical reactivity of free radicals generated from such crosslinkable functional groups under irradiation or heat exposure. In one embodiment, the cross-linking agent comprises a functional group capable of forming a secondary or tertiary aliphatic or cycloaliphatic radical. In another alternate embodiment, the cross-linking agent comprises a functional group capable of forming an aromatic radical, for example a benzyl radical. Other crosslinkable functional groups include methacrylates, acrylates, acrylamides, vinylketones, styrenics, vinyl ethers, vinyl groups, allyl groups, benzyl groups, and groups containing tertiary carbon-hydrogen bonds, for example isobutyl groups.

Suitable cross-linking agents 37 include but are not limited to methacrylates, acrylates and vinyl ketone reagents. These reagents can be covalently bound to a binder material or form crosslinked polymers themselves upon exposure to high energy irradiation or heat. For example, suitable cross-linking agents include without limitation the reagents acryloyl chloride, (2E)-2-butenoyl chloride, maleic anhydride, 2(5H)-furanone, methyl acrylate, 5,6-dihydro-2H-pyran-2-one, ethyl acrylate, methyl crotonate, allyl acrylate, vinyl crotonate, 2-isocyanatoethyl methacrylate, methacrylic acid, methacrylic anhydride, methacryloyl chloride, glycidyl methacrylate, 2-ethylacryloyl chloride, 3-methylenedihydro-2(3H)-furanone, 3-methyl-2(5H)-furanone, methyl 2-methylacrylate, methyl trans-2-methoxyacrylate, citraconic anhydride, itaconic anhydride, methyl (2E)-2-methyl-2-butenoate, ethyl 2-methylacrylate, ethyl 2-cyanoacrylate, dimethylmaleic anhydride, allyl 2-methylacrylate, ethyl (2E)-2-methyl-2-butenoate, ethyl 2-ethylacrylate, methyl (2E)-2-methyl-2-pentenoate, 2-hydroxyethyl 2-methylacrylate, methyl 2-(1-hydroxyethyl)acrylate, 3-(methacryloyloxy)propyltrimethoxysilane, 3-(diethoxymethylsilyl)propyl methacrylate, 3-(trichlorosilyl)propyl 2-methylacrylate, 3-(trimethoxysilyl)propyl 2-methylacrylate, 3-tris(trimethylsiloxy)silylpropyl methacrylate, 6-dihydro-1H-cyclopenta(c)furan-1,3(4H)-dione, methyl 2-cyano-3-methylcrotonate, trans-2,3-dimethylacrylic acid, and N-(hydroxymethyl)acrylamide.

Suitable vinyl and allyl reagents which may serve as a cross-linking agent include, without limitation, allyl bromide, allyl chloride, diketene, 5-methylenedihydro-2(3H)-furanone, 3-methylenedihydro-2(3H)-furanone, 2-chloroethyl vinyl ether, and 4-methoxy-2(5H)-furanone.

Suitable isocyanate reagents which may serve as cross-linking agent include, without limitation, vinyl isocyanate, allyl isocyanate, furfuryl isocyanate, 1-ethyl-4-isocyanatobenzene, 1-ethyl-3-isocyanatobenzene, 1-(isocyanatomethyl)-3-methylbenzene, 1-isocyanato-3,5-dimethylbenzene, 1-bromo-2-isocyanatoethane, (2-isocyanatoethyl)benzene, 1-(isocyanatomethyl)-4-methylbenzene, 1-(isocyanatomethyl)-3-methylbenzene, 1-(isocyanatomethyl)-2-methylbenzene, and the like.

Suitable styrenic reagents which may serve as a cross-linking agent include, without limitation, 3-vinylbenzaldehyde, 4-vinylbenzaldehyde, 4-vinylbenzyl chloride, trans-cinnamoyl chloride, phenylmaleic anhydride, 4-hydroxy-3-phenyl-2(5H)-furanone, and the like.

Suitable epoxide reagents which may serve as the cross-linking agent 37 include, without limitation, glycidyl methacrylate, glycidyl vinyl ether, 2-(3-butenyl)oxirane. 3-vinyl-7-oxabicyclo[4.1.0]heptane, limonene oxide, and the like.

In some embodiments, the cross-linking agent 37 may include multiple (e.g., two, three, or more than three) different types of functional groups that may facilitate formation of the fluid capture material 44. In general, the cross-linking agent 37 may include a first functional group that reacts with the binder material 36 and a second functional group that may cross-link. For example, the cross-linking agent 37 may include an anhydride functional group and an acrylate functional group, an epoxide functional group and an acrylate functional group, an isocyanate functional group and a methacrylate functional group, and the like. As one non-limiting example, the binder material 36 may include poly(vinyl alcohol) and the cross-linking agent 37 may include 2-isocyanato ethylmethacrylate (2-IEM), which includes both an isocyanate functional group and a methacrylate functional group. As another non-limiting example, the binder material 36 may include poly(vinyl butyral) and the cross-linking agent 37 may include 2-(3,4-epoxycyclohexyl)ethyl-trimethoxysilane.

Generally, the pore precursor 35 may be any suitable pore precursor known in the art that facilitates the system according to the present disclosure. As used herein, a “pore precursor” is a precursor material provided in the fluid capture material 44 that is removed from the fluid capture material 44 to leave at least one pore in its place. The pore precursor allows control of the porosity of the fluid capture material 44 and results in enhanced porosity. Enhanced porosity subsequently results in enhanced diffusion of a gas within the fluid capture material 44.

Enhanced porosity may be present in the form of at least one of an increased average porosity, increased porosity near the substrate, increased porosity near an interface between air and the fluid capture material 44, and increased porosity near an edge of fluid capture material 44.

Enhanced porosity is provided by at least one pore formed from the pore precursor. The at least one pore is of sufficient size and shape to result in enhanced gas diffusion.

Useful sizes and shapes of pores depend on film thickness, sorbent size, sorbent shape, and other related factors. In some embodiments, the at least one pore has an average cross-sectional area in a range of from about 5 μm to about 500 μm.

In some embodiments, the at least one pore has an average cross-sectional shape selected from the group consisting of symmetrical shapes, asymmetrical shapes, amorphous shapes, spheres, cylinders, cubes, octahedra, needles, and combinations thereof.

In some embodiments, enhanced porosity results in decreased film density.

Generally, porosity may be measured according to any suitable means known in the art. In some embodiments, porosity is measured via ellipsometry. The percent porosity within an MOF thin film may be calculated according to the following equation:

$$\% Porosity = \frac{{n}_{fram , A , V}^{2} - {n}_{fram , V}^{2}}{{n}_{a}^{2} - {n}_{v a c}^{2}} \times 100 \%$$

where η_{fram,A,V} and η_{fram,V} are volume weighted averages of refractive indices of the MOF films with and without solvent in-filtration respectively; and
ηₐ is the refractive index measured of the film infiltrated with high-boiling point solvent (which is often dimethylformamide) and η_{vac} is the refractive index of vacuum and estimated as 1.

In some embodiments, the pore precursor is selected from the group consisting of waxes, salts, non-reactive gas-generating species, thermally labile polymers, and combinations thereof.

In some embodiments, the pore precursor has an average cross-sectional shape selected from the group consisting of symmetrical shapes, asymmetrical shapes, amorphous shapes, spheres, cylinders, cubes, octahedra, needles, and combinations thereof.

In some embodiments, the pore precursor is non-reactive. Non-reactive pore precursors do not react with binder material 36, sorbent-binder material 38, or cross-linking agent 37. When pore precursors react with any of binder material 36, sorbent-binder material 38, and cross-linking agent 37, they become irremovable from the fluid capture material 44.

In some embodiments, the pore precursor is a wax having a low viscosity, such that the wax melts upon heating and flows out of the fluid capture material 44 without assistance.

In some embodiments, the pore precursor is a modified wax. In some embodiments, the pore precursor is a wax modified with a block copolymer. The modified wax has a modified size or shape compared to an unmodified wax.

In some embodiments, the pore precursor is a wax that is insoluble in a slurry or solvent blend according to the present disclosure. In some embodiments, the pore precursor is a wax that is soluble in the slurry or solvent blend, such that the wax phase-separates upon drying. In some embodiments, the pore precursor is a wax selected from the group consisting of dicetyl fumarate, paraffin, dimethyl eicosanedioate, and combinations thereof.

In some embodiments, the pore precursor is a salt dissolvable in a solvent. The solvent is applied to the fluid capture material 44 containing the salt, which causes the salt to dissolve in the solvent and flow out of the fluid capture material 44.

In some embodiments, the pore precursor is a non-reactive gas-generating species, wherein the non-reactive gas is selected from the group consisting of N₂, CO₂, H₂, and combinations thereof.

In some embodiments, the pore precursor is a non-reactive gas-generating species selected from the group consisting of azo-containing compounds, azobisisobutyronitrile ([(CH₃)₂C(CN)]₂N₂), carbamate salts, μ-keto carboxylic acids, and combinations thereof.

Generally, the pore precursor may be any suitable f-keto carboxylic acid known in the art that facilitates the sorbent described herein. In some embodiments, the pore precursor is a β-keto carboxylic acid selected from the group consisting of acetoacetic acid, malonic acid, sodium malonate dibasic, sodium malonate dibasic monohydrate, lithium acetoacetate, methylmalonic acid, monomethyl malonate, methyl potassium malonate. 1,3-acetonedicarboxylic acid, dimethylmalonic acid, methyl acetoacetate, mono-ethyl malonate, ethyl potassium malonate, ethylmalonic acid, oxaloacetic acid, mono-tert-butyl malonate, butylmalonic acid, diethylmalonic acid, cyclopropane-1,1-dicarboxylic acid, cyclobutane-1,1-dicarboxylic acid, dihydroxyfumaric acid hydrate, cyclohexane-1,1-dicarboxylic acid, (1S)-(+)-ketopinic acid, 4-hydroxy-6-methyl-2-pyrone, 2-oxo-3-piperidinecarboxylic acid, and combinations thereof.

In some embodiments, one or more additives may be added to form the sorbent-binder material 38. For example, the additives may include dispersants to facilitate forming a suspension, such as anionic dispersants, cationic dispersants, non-ionic dispersants, defoamers, wetting agents, adhesion promoters, or any combination thereof. For example, suitable anionic dispersants may include polymeric alkoxylate or phosphate ester. For example, suitable non-ionic dispersants may include polyurethane. For example, suitable cationic dispersants may include polyoxyethylene fatty ammonium sulfate. In general, the amount of dispersant added may be less than the amount of the binder material 36. For example, the sorbent-binder material 38 may include 10% by weight of binder material 36 and 0.5% by weight of dispersant, 1% by weight of dispersant, or greater than 1% by weight of dispersant. As another non-limiting example, the sorbent-binder material 38 may include 15% by weight of binder material 36 and 1% by weight of dispersant. 3% by weight of dispersant, or greater than 5% by weight of dispersant. As another non-limiting example, the sorbent-binder material 38 may include 13% by weight of binder material 36 and 1% by weight of dispersant, 3% by weight of dispersant, or greater than 5% by weight of dispersant. For example, in an example sorbent-binder material 38 where the binder material 36 is aminopropylsilsesquioxane, the binder material 36 may be formed using a binder solution having 13% binder and 2% dispersant. The dispersant may include polyethyleneimine (PEI), such as PEI-low (e.g., M_{W} between approximately 20,000 and 25,000 g/mol, and Mₙ between approximately 8,000 to 12,000) or PEI-high (e.g., M_{W} between approximately 70,000 and 80,000 g/mol, and M_{W} between approximately 55,000 to 65,000).

At block 40, the sorbent-binder material 38 is deposited onto, applied to, formed integrally with (e.g., during manufacture), or otherwise coupled to the substrate 16, such as to one or more surfaces of the substrate 16, thereby forming a fluid capture coated substrate 42. In some embodiments, the substrate may include certain metal substrates (e.g., aluminum, titanium) or 3-D printed metal substrates. For example, the substrate 16 may include a fluid contactor with a metal surface. In some embodiments, the substrate 16 comprises metal alloys (e.g. Inconel or stainless steel). As referred to herein, a “fluid contactor” or “direct fluid contactor” refers to a structure configured to receive a fluid flow, and the structure may include a porous and/or semi-porous material, such that a portion of the fluid flow may permeate through the fluid contactor. In some embodiments, the fluid flow may include an ambient air flow. In some embodiments, the fluid flow may include a flue gas flow or an exhaust gas flow from power generating equipment (e.g., a gas turbine). As such, the binder material 36 may be selected to have a relatively high binding to the metal surface.

In some embodiments, the substrate 16 can be a polymer or polymer composite. Polyolefins (e.g., polyethylene, polypropylene, polymethylpentene, polystyrene, substituted polystyrenes, poly(vinyl chloride) (PVC), polyacrylonitriles), polyamide, polyester, polysulfone, polyether, acrylic and methacrylic polymers, polystyrene, polyurethane, polycarbonates, polyesters (e.g., polyethylene terephthalic ester, polybutylene terephthalic ester), polyether sulfones, polypropylene, polyethylene, polyphenylene sulfone, cellulosic polymer, polyphenylene oxide, polyamides (e.g., nylon, polyphenylene terephthalamide), and combinations of two or more of the foregoing polymers may be utilized as substrates. Fluoropolymers, which may be used as the substrate include, without limitation, ePTFE, polyvinylidene difluoride (PVDF), poly(tetrafluoroethylene-co-hexafluoropropylene (FEP), poly(ethylene-alt-tetrafluoroethylene) (ETFE), polychlorotrifluoroethylene (PCTFE), poly(tetrafluoroethylene-co-perfluoropropyl vinyl ether) (PFA), poly(vinylidene fluoride-co-hexafluoropropylene (PVDF-co-HFP), and polyvinyl fluoride (PVF).

In general, depositing the sorbent-binder material 38 onto the substrate 16 may include curing the sorbent-binder material 38, which includes the cross-linking agent 37, thereby forming a fluid capture material 44 or coating that is a polymer and sorbent composite material. Put differently, the fluid capture material 44 refers to the sorbent-binder material 38 where the binder material 36 is cross-linked via one or more cross-linking agents 37. As described herein, cross-linking the sorbent-binder material 38 may provide a material and/or coating (i.e., the fluid capture material 44) that has relatively higher structural integrity as compared to not cross-linking the sorbent-binder material 38. Further, cross-linking the sorbent-binder material 38 may provide a material and/or coating that has a relatively higher binding capacity to fluids.

It should be noted that, at least in some instances, the sorbent-binder material 38 may be deposited multiple times on the substrate 16. It is presently recognized that, at least in some instances, depositing a relatively thick layer (e.g., greater than 1 mm, greater than 2 mm, or greater than 5 mm) may result in fluid capture material 44 (e.g., a fluid capture material or a fluid capture coating) having one or more cracks. Therefore, to facilitate reducing, preventing, or mitigating cracking (e.g., mudcracking), it may be advantageous to deposit multiple layers to ultimately form the fluid capture material 44 having a desired thickness (e.g., between 0.1 mm and 0.9 mm, between 1.1 mm and 1.3 mm, between 0.1 and 2.0 mm, between 2.5 mm and 3.5 mm). For example, the fluid capture material 44 may include 3 layers and have a total thickness of 1.2 mm. As another non-limiting example, the fluid capture material may include 6 layers and have a total thickness of 3 mm. For example, to deposit multiple layers, the process 30 may include depositing a first amount of the sorbent-binder material 38, curing the first amount of the sorbent-binder material to form a first layer, and repeating the process one or more times to form one or more additional layers, thereby forming a fluid capture material having multiple layers (e.g., 2, 3, 4, 5, 6, 7). In some embodiments, the first layer of the fluid capture material 44 may be pre-wetted before adding a second layer. In general, pre-wetting includes providing a suitable solvent to first layer, such as toluene, ethanol, water or a combination thereof. After pre-wetting the first layer, a second layer may be formed on top of the pre-wet first layer. In general, the second layer may be formed in a generally similar manner as described with respect to the first layer.

In some embodiments, the total thickness of the fluid capture material or coating may be less than 1 mm. For example, the total thickness may be between 0.1 mm and 0.9 mm, 0.2 mm and 0.8 mm, 0.2 and 0.7 mm, 0.3, and 0.6 mm, or between 0.4 mm and 0.5 mm. In some embodiments, each layer of the fluid capture material 44 may have the same thickness, such that the thickness formed for each layer (e.g., as described with respect to FIG. 2) is the total thickness/n, where “n” is the number of layers formed. In some embodiments, one or more layers of the fluid capture material 44 may have a different thickness. For example, each subsequently formed layer may have a thinner thickness than a preceding layer. Alternatively, each subsequently formed layer may have a thicker thickness than a preceding layer.

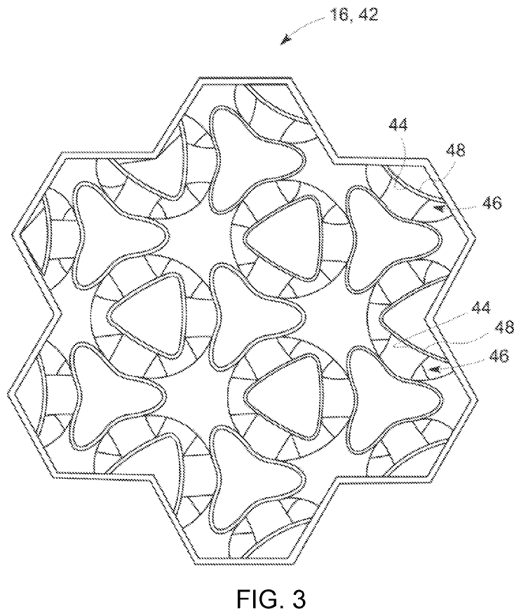

As described herein, the fluid capture material 44 may be deposited onto one or more surfaces of the substrate 16, such as an air contactor. To illustrate this, FIG. 3 shows a cross-sectional diagram of a substrate 16 including the fluid capture material 44 (i.e., a fluid capture coating substrate 42). In the illustrated embodiment, the substrate 16 is material formed using additive printing. Further, as illustrated, the fluid capture material 44 includes one or more channels 46 that generally permeate through a portion of the fluid capture material 44. In general, the sorbent material 34 may be capable of forming a porous material. Accordingly, the one or more channels 46 may also form in the fluid capture material 44.

As illustrated, each channel 46 generally includes a wall 48 that has the fluid capture material 44 bound to its surface. As such, a gas flow that flows through the channels of the fluid capture coated substrate 42 may contact the fluid capture material 44, and thus, facilitate the binding of a target fluid (e.g., a CO₂) with the fluid capture material 44.

At block 41, the pore precursor 35 is removed 41 from the fluid capture material 44 to form at least one pore in the fluid capture material 44. Generally, the pore precursor 35 may be removed 41 from the fluid capture material 44 by any suitable means known in the art that facilitates the system according to the present disclosure. In some embodiments, the pore precursor 35 is removed 41 from the fluid capture material 44 by a technique selected from the group consisting of heating, washing, solvent extraction, and combinations thereof.

As described herein, the disclosed fluid capture material 44 may have a relatively high fluid-binding capacity (e.g., water capacity and/or CO₂ capacity). Table 1 shows results of CO₂ capacity measurements for certain substrates coated with a fluid capture material 44. In general, the fluid capture materials 44 corresponding to Table 1 were doctor blade coated onto 2 in.×2 in. Inconel 718 coupons and evaluated for CO₂ capture performance (e.g., CO₂ capacity) at 0.04 kPa. A sampling of MOF-binder composites have been evaluated in aluminum weighing pans to establish film curing conditions, preliminary structural integrity of the films, and ambient sorption measurements. An example process for coating coupons with the slurry (i.e., the sorbent-binder material 38) entails mixing a MOF powder (i.e., a sorbent material 34) with an appropriate binder material 36, wetting agent, additive, and solvent in a container. The mixture may be vortexed for 1-2 min and then sonicated in an ultrasonic bath for 20 min at 72 kHz. The slurry is then coated onto the substrate 16 using a doctor blade of the appropriate gap (10-50 mil, 254-1270 μm) and left to dry in ambient conditions. For coatings in aluminum pans, the slurry may be added to the pan using a plastic pipette, the pan may be tilted to cover the bottom, and left to dry in ambient conditions. Once dry, pans or coupons are cured and activated using the appropriate conditions.

Further aspects of the present disclosure are provided by the subject matter of the following clauses:

1. A system, comprising:
a substrate; and
a fluid capture material formed on one or more surfaces of the substrate, wherein the fluid capture material comprises:
   a sorbent material configured to bind one or more fluids comprising water, carbon dioxide, sulfur oxides, alcohols, or a combination thereof; and
   one or more binder materials, wherein the binder material is optionally at least partially cross-linked;
   wherein the fluid capture material comprises at least one pore.

2. The system of the preceding clause, wherein the fluid capture material comprises less than 15% by weight of the one or more binder materials.

3. The system of any preceding clause, wherein the sorbent material comprises a metal-organic framework (MOF), a covalent-organic framework (COFs), polymeric resins, silicas, zeolites, or a combination thereof.

4. The system of any preceding clause, comprising a cross-linking agent, wherein the binder material is at least partially cross-linked with the crosslinking agent, and wherein the cross-linking agent comprises one or more of a methacrylate reagent, an acrylate reagent, a vinyl ketone reagent, a vinyl reagent, or an allyl reagent.

5. The system of any preceding clause, comprising a cross-linking agent, wherein the binder material is at least partially cross-linked with the crosslinking agent, and wherein the cross-linking agent comprises polyacrylic acid.

6. The system of any preceding clause, wherein the one or more binder materials comprise vinyl polymers, starch, alkyl cellulose, or a combination thereof.

7. The system of any preceding clause, comprising a cross-linking agent, wherein the binder material is at least partially cross-linked with the crosslinking agent, and wherein a ratio of the cross-linking agent to the binder material is less than 25%.

8. The system of any preceding clause, wherein a thickness of the fluid capture material is between 0.1 and 3.5 mm.

9. The system of any preceding clause, wherein the fluid capture material comprises greater than 80% by weight of the sorbent material.

10. The system of any preceding clause, where the binder material that is at least partially cross-linked comprises a cross-linked density that is greater than 10%.

11. A method, comprising:
providing a sorbent material configured to bind one or more fluids comprising water, carbon dioxide, sulfur oxides, alcohols, or a combination thereof;
providing one or more binder materials, wherein the one or more binder materials optionally comprises a component capable of forming a cross-linked polymer;
optionally providing a cross-linking agent;
providing a pore precursor;
generating a sorbent-binder material based on the sorbent material, the one or more binder materials, optionally the cross-linking agent, and the pore precursor;
applying the sorbent-binder material to a substrate;
forming a fluid capture material using the sorbent-binder material applied to the substrate; and
removing the pore precursor to form at least one pore in the fluid capture material.

12. The method of the preceding clause, wherein forming the fluid capturing material comprises:
forming a first layer of the fluid capture material using the sorbent-binder material;
pre-wetting the first layer; and
forming a second layer onto the pre-wet first layer.

13. The method of any preceding clause, wherein the one or more binder materials comprise a first binder material and a second binder material, wherein the first binder material is distinct from the second binder material

14. The method of any preceding clause, wherein providing one or more binder materials comprises providing a first amount of the one or more binder materials, wherein providing the cross-linking agent comprises providing a second amount of the cross-linking agent, and wherein the ratio of the second amount to the first amount is less than 1/3.

15. The method of any preceding clause, wherein providing one or more binder materials comprises providing a first amount of the one or more binder materials, wherein providing the cross-linking agent comprises providing a second amount of the cross-linking agent, and wherein the ratio of the second amount to the first amount is less than 1/4.

16. A system, comprising:
a fluid capture material configured to bind one or more fluids, wherein the fluid capture material comprises:
   a sorbent material configured to bind one or more fluids comprising water, carbon dioxide, sulfur oxides, alcohols, or a combination thereof; and
   one or more binder materials;
wherein the fluid capture material comprises at least one pore;
wherein the one or more binder materials are optionally at least partially cross-linked; and
a fluid contactor having one or more surfaces coated with the fluid capture material.

17. The system of the preceding clause, wherein a thickness of the fluid capture material is between 0.1 mm and 2.0 mm on at least one surface of the one or more surfaces.

18. The system of any preceding clause, wherein a thickness of the fluid capture material is between 0.5 mm and 1.5 mm on at least one surface of the one or more surfaces.

19. The system of any preceding clause, wherein the fluid capture material comprises greater than 80% by weight of the sorbent material.

20. The system of any preceding clause, where the binder material that is at least partially cross-linked comprises a cross-linked density that is greater than 50%.

### EXAMPLES

Without further elaboration, it is believed that one skilled in the art using the preceding description can utilize the present invention to its fullest extent. The following Examples are, therefore, to be construed as merely illustrative, and not limiting of the disclosure in any way whatsoever. The starting material for the following Examples may not have necessarily been prepared by a particular preparative run whose procedure is described in other Examples. It also is understood that any numerical range recited herein includes all values from the lower value to the upper value. For example, if a range is stated as 10-50, it is intended that values such as 12-30, 20-40, or 30-50, etc., are expressly enumerated in this specification. These are only examples of what is specifically intended, and all possible combinations of numerical values between and including the lowest value and the highest value enumerated are to be considered to be expressly stated in this application.

### Fluid Capture Materials.

TABLE 1
CO₂ capacity of certain fluid capture materials

| | | | CO₂ capacity | | |
|---|---|---|---|---|---|
| | | | (mmol/g MOF) | | |
| | | | 20% | 50% | 75% |
| Example | Sorbent | Binder | RH | RH | RH |
| 1 | MOF-808-Gly | None - powder, | 0.3 | | |
| | | no coupon | | | |
| 2 | MOF-808-Gly | aminopropyl- | 0.37 | | |
| | | silsesquioxane | | | |
| 3 | MOF-808-Gly | PVA/PAA | | | 0.38 |

Table 1 shows examples of fluid capture materials 44 that may be used to capture CO₂. Generally, table 1 shows the CO₂ capacity of a control (e.g., example 1) as compared to samples that include a fluid capture material 44 formed using a sorbent material (i.e., MOF-808-Gly) and a binder material (e.g., examples 2 and 3) that is capable of cross-linking. More specifically, example 1 includes sorbent material. MOF-808-Gly, in powder form, without being deposited on a coupon. The CO₂ capacity of example 1 at 400 ppm CO₂ in N₂ at 20° C. and 20% RH is 0.3 mmol/g.

Examples 2 and 3 illustrate fluid capture materials 44 formed using a sorbent material and a binder material that is, ultimately, cross-linked. More specifically, example 2 is a fluid capture material 44 with a sorbent material 34 (e.g., MOF-808-Gly) and a binder material 36 (e.g., aminopropylsilsesquioxane) that is capable of cross-linking. To prepare example 2, a slurry was prepared by mixing 2.44 g of a 25% aqueous solution of aminopropylsilsesquioxane, 17.6 g deionized water. 0.12 g of Triton™ X-100, and 5.1 g of MOF-808-Gly. After mixing, the slurry was coated on 2″ by 2″ Inconel coupons, dried, and cured overnight at 120° C. under vacuum. A high-quality coating was obtained that had an equilibrium CO₂ uptake (e.g., CO₂ capacity) of 0.37 mmol/g when exposed to 400 ppm CO₂ in N₂ gas stream at 20° C. and 20% RH.

Example 3 is a fluid capture material 44 with a sorbent material 34 (e.g., MOF-808-Gly), a binder material 36 (e.g., PVA), and a cross-linking agent 37 (e.g., PAA). To prepare example 3, a slurry was prepared by mixing 1.55 g of an aqueous solution of 15% PVA (e.g., 88% hydrolyzed) and 3% PAA, 5.2 g deionized water, ~3 mg of Triton™ X-100, and 2.5 g of MOF-808-Gly. After mixing, the slurry was coated on 2″ by 2″ Inconel coupons, dried, and cured overnight at 125° C. under vacuum. The fluid capture material was obtained which scored a 3B on the ASTM D3359-17 adhesion test and which had an equilibrium CO₂ uptake of 0.38 mmol/g when exposed to 400 ppm CO₂ in N₂ gas stream at 20° C. and 75% RH. In general, examples 2 and 3 illustrate two cross-linked aqueous binder formulations utilized with MOF-808-Gly to form a fluid capture material 44, which have CO₂ binding capacity that is approximately equal to that of example 1. Further, examples 2 and 3 of the fluid capture materials have good adhesion to a substrate.

In some embodiments, the fluid capture material 44 may be formed using non-aqueous solvents. For example, another example (i.e., example 4) of a fluid capture material 44 generally includes a sorbent material 34 (e.g., MOF-808-Gly) and a silicon-containing binder material 36 capable of cross-linking. First, 1.2 mL of a 0.2 g/mL solution of SPR100 in methyl ethyl ketone (MEK) was mixed with 94 mg of disilanol PDS-1615, 53 μL alkoxysilane SIB1140.0, and 69 mg Hypermer™-KD1 in a vial. Separately, 3.0 g MOF-808-Gly was mixed with 5 mL isopropanol (IPA). The SPR100 containing solution was added to the MOF-808-Gly/IPA suspension. The SPR100 vial was rinsed with 2×0.5 mL MEK and added to the combined mixture. The slurry was further diluted with 2 mL IPA to obtain a viscosity amenable to coating. Then. 38 μL of trihexylamine was added to this slurry and the mixture was coated on 2″ by 2″ Inconel coupons, dried, and cured at 90° C. under vacuum for 1 hr. A high-quality coating was obtained which scored a 4A on the ASTM D3359-17 adhesion test.

As described above, the fluid capture material 44 may be capable of binding water in certain embodiments. Several examples of fluid capture materials 44 in accordance with the present disclosure, as well as the performance of such fluid capture materials 44 are described below.

A first example of a water-binding fluid capture material 44 may include a sorbent material 34 (i.e., MOF-303), a binder material 36 (i.e., PVA), and a cross-linking agent (i.e., PAA) deposited on a metal substrate. More specifically, the first example of the water-binding fluid capture material 44 may be prepared by forming a slurry via mixing 0.56 g of an aqueous solution of 15% poly(vinyl alcohol) [PVA, 88% hydrolyzed] and 3% poly(acrylic acid) [PAA], 2.0 g deionized water, ~3 mg AGITAN 351, 1.0 g of MOF-303 and 0.02 g Tergitol 15-S-7. After mixing, the slurry was coated on 2″ by 2″ Inconel coupons and cured overnight at 125° C. A high-quality coating was obtained that was well adhered and had an equilibrium water uptake of 26-28% by weight when tested in a humidity chamber set at 20% RH and 25° C.

A second example of a water-binding fluid capture material 44 includes a sorbent material 34 (e.g., MOF-303), a binder material 36 (e.g., PVA), and a cross-linking agent (e.g., PAA) deposited on a glass filled nylon coupon (e.g., a glass filled nylon substrate). More specifically, the second example of the water-binding fluid capture material 44 may be prepared by forming a similar slurry as described with respect to the first example of the water-binding fluid capture material 44 above and coating the slurry on a 2″ by 2″ glass-filled polyamide (PA12) nylon coupon. The coated sample was dried at room temperature and then cured overnight at 120° C. Once cooled to room temperature the sample was immersed in water to release air bubbles and then patted dry. A second layer of slurry was then coated as before. This process was repeated an additional time. After the final cure at 120° C. the coating weighed 0.9216 g, and adhered well to the substrate. The equilibrium water uptake at 20% RH/25° C. was 28% by weight.

A third example of water-binding fluid capture material 44 includes multiple binder materials 36. For example, the third example of the water binding fluid capture material 44 may include binder materials 36 such as PVA. PAA, and poly(methyl/phenylsilsesquioxane). More specifically, the third example of the water-binding fluid capture material 44 may be prepared by mixing 1.78 g of an aqueous solution of 7.5% PVA [80% hydrolyzed] and 1.5% PAA with 3.5 g deionized water, 0.02 g DISPERBYK 190, ~3 mg AGITAN 351, and 2.0 g of MOF-303. A solution of 0.08 g Wacker MP-50E silicone emulsion diluted with 0.5 g deionized water was added to this mixture. After mixing, the slurry was coated on a 2″ by 2″ glass-filled PA12 nylon coupon. After drying at room temperature, the samples were cured at 120° C. for 4 hours. After cooling, the sample was immersed in water to release air bubbles, patted dry and then coated with another layer of slurry. The drying/curing process was then repeated as before. Two more layers of slurry were then coated on top of the first two using the same procedure. The weight of dried/cured coating at the end of this process was 1.4946 g. The coating was well adhered and had no cracks. The equilibrium water uptake at 20% RH/25° C. was 31-32% by weight.

A fourth example of a water-binding fluid capture material 44 includes a sorbent material 34, such as MIL-160. To prepare the fourth example of the water-binding fluid capture material, 2.44 g of an aqueous solution of 13.5% PVA [88% hydrolyzed] and 4.5% PAA were mixed with 5.9 g deionized water, 0.040 g DISPERBYK 190, 0.030 g AGITAN 351, 4.34 g MIL-160 and 0.050 g Tergitol 15-S-7. After mixing, the slurry was coated on a 2″ by 2″ Inconel coupon. The sample was dried at room temperature and then overnight at 120° C. After cooling the sample was immersed in water to release bubbles and then was patted dry. A second layer of slurry was applied and cured as before. The second layer did not adhere to the first layer and subsequently flaked off.

A fifth example of a water-binding fluid capture material 44 includes multiple binder materials 36, such as silicon-containing binder materials, PVA, and PAA. It is presently recognized that utilizing hybrid binder materials 36 (i.e., two, three, four, or more than four different or distinct binder materials) may improve the adherent properties of the fluid capture material 44 or layer to a substrate and/or the adherent properties for each layer of multi-layer coatings. To prepare the fifth example of the water-binding fluid capture material, 8.0 g of an aqueous solution of 7.5% PVA [80% hydrolyzed) and 1.5% PAA were mixed with 9.0 g deionized water, 0.10 g DISPERBYK 2055, 0.015 g AGITAN 351, and 8.0 g of MIL-160. To this was added a solution of 0.08 g Wacker MP-50E silicone emulsion diluted with 2.0 g deionized water. After mixing, this slurry was used to coat a small Inconel heat exchanger. After drying at room temperature, the samples were cured at 120° C. for 2 hours. After cooling, the sample was immersed in water to release air bubbles, patted dry and then coated with another layer of slurry. The drying/curing process was then repeated as before. Finally, a third layer was applied as before. After final curing overnight at 120° C., 3.1 g of well adhered coating was obtained. The equilibrium water uptake at 20% RH/25° C. was 30-32% by weight.

It is further recognized that cross-linking the composite coating may improve the structural integrity of the fluid capture material 44 or coating. To illustrate the improved structural integrity based on the addition of a cross-linking agent 37, two compositions of sorbent materials 34 and binder materials 36 were prepared. The first composition is in accordance with the disclosed fluid capture material 44, and thus is formed by cross-linking the binder material 36 (i.e., via addition of PAA). In the second composition, the binder material 36 is not cross-linked (i.e., no PAA was added). To prepare the first composition, a slurry was prepared by mixing 0.56 g of an aqueous solution of 13.5% poly(vinyl alcohol) [PVA, 88% hydrolyzed] and 4.5% poly(acrylic acid) [PAA], 1.4 g deionized water, 0.02 g DISPERBYK 190, and 1.0 g of MIL-160. After mixing, the slurry was coated on a 1″ by 1″ Inconel coupon, dried at room temperature and cured overnight at 125° C. in a vacuum oven. The coupon was cooled to room temperature in a vacuum desiccator and then quickly weighed. It was then submersed in 10 mL of deionized water and put in a 90° C. oven for 2 hours. At the end of this time the coupon was removed and dried at 90° C. for an hour followed by 2 hours in the 125° C. vacuum oven. Finally, the sample was cooled in a vacuum desiccator and reweighed as before. The weights were: (1) uncoated coupon: 5.0038 g; (2) coated coupon after cure: 5.3206 g (i.e., the coating weight was 0.3168 g): (3) coated coupon after water immersion/drying: 5.3087 g (i.e., the coating was 0.3049 g); and (4) coating weight retained after water immersion: 96.2%.

To prepare the second composition (i.e., prepared without using a cross-linking agent 37), a slurry was prepared by mixing 0.67 g of an aqueous solution of 15% poly(vinyl alcohol) [PVA, 88% hydrolyzed], 1.3 g deionized water, 0.02 g DISPERBYK 190, and 1.0 g of MIL-160. After mixing, the slurry was coated on a 1″ by 1″ Inconel coupon, dried at room temperature and cured overnight at 125° C. in a vacuum oven. The coupon was cooled to room temperature in a vacuum desiccator and then quickly weighed. It was then submersed in 10 mL of deionized water and put in a 90° C. oven for 2 hours. Soon after immersion in water the coating started to fall apart and come off the coupon. At the end of this time the coupon was removed and dried at 90° C. for an hour followed by 2 hours in the 125° C. vacuum oven. Finally, the sample was cooled in a vacuum desiccator and reweighed as before. The weights were: (1) uncoated coupon: 5.0320 g: (2) coated coupon after cure: 5.1974 g (i.e., the coating weight was 0.1654 g); (3) coated coupon after water immersion/drying: 5.0573 g (i.e., the coating was 0.0253 g); and (4) coating weight retained after water immersion: 15.3%. In particular, the first composition (i.e., the example of the fluid capture material 44 including a cross-linked binder) contains PAA and the cured film obtained in this case retained 96% of its mass after 2 hours in 90° C. water. In contrast, using the second composition (i.e., when PVA was used without any cross-linker), only 15% of the mass was retained after testing the same way.

As described herein, the fluid capture material 44 may be formed using a cross-linking agent 37 that has different types of functional groups that may facilitate formation of the fluid capture material 44. To prepare an example of such a composition, 0.30 g poly(vinyl butyral) was dissolved in 6.0 g of isopropanol. Further, 0.065 g 2-(3,4-epoxycyclohexyl)ethyl-trimethoxysilane, 3.0 g of an amine treated silica sorbent, and 0.07 g BYK9076 were then mixed in. The resulting slurry was coated on aluminum coupons using a doctor blade. After drying at room temperature, the samples (e.g., the aluminum coupons coated with the slurry) were placed in a 90° C. oven for an hour to cure. The CO₂ uptake was measured under dry conditions at 25 C using 400 ppm CO₂ in nitrogen. The average value was determined to be 0.734 mol CO₂/kg coating (0.032 g/g).

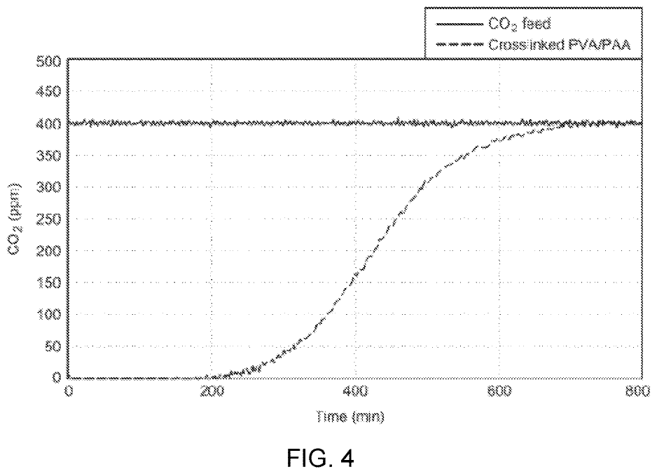

FIG. 4 is a graph having a y-axis corresponding to an amount of CO₂ (ppm) and an x-axis corresponding to time (minutes (min)). In this example, the fluid capture material 44 was formed using a binder material 36 including PVA/PAA as described for example 3 in Table 1. Further, the fluid capture material 44 was subject to a fluid flow at 50 standard cubic centimeters per minute (sccm) having 400 ppm of CO₂ and 75% RH. As generally shown in the graph, CO₂ was detected after about 170 min of flowing the fluid flow into the fluid capture material or coating.

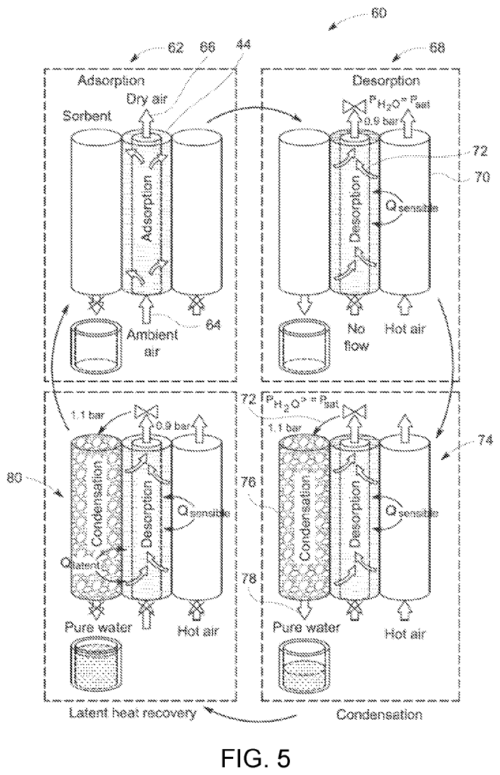

As described herein, the fluid capture material 44 may be capable of capturing a target fluid, such as H₂O. In such embodiments, it is presently recognized that it may be advantageous to form a fluid capture material 44 that is capable of releasing the capture fluid. To illustrate this, FIG. 5 is a diagram illustrating a method 60 for capturing a target fluid (e.g., the target fluid 18 as described with respect to FIG. 1) and subsequently releasing the target fluid in a controlled manner (i.e., when it may be desirable to remove the target fluid 18. For example, in an embodiment where the target fluid 18 includes water, it may be desirable to utilize the disclosed fluid capture material 44 to extract water from a fluid source, such as air having a relatively high moisture content (e.g., greater than 500 ppm of water), and subsequently releasing water, thereby producing pure water.

Referring to the method 60, at block 62, a gas flow 64 is provided to the substrate 16 coated with the fluid capture material 44. Water in the gas flow 64 binds to the capture coating, thereby generating a dry gas flow 66. At block 68, a heat exchanger 70 is heated (e.g., using a hot air at a temperature greater than 80° C., greater than 85° C., greater than 90° C., or greater than 95° C.). In any case, the water bound to the fluid capture material 44 may be released as steam 72. At block 74, a condenser 76 may receive the steam 72 and cool the steam 72, thereby producing water 78. At block 80, the heat may be recovered. In this way, the fluid capture material 44 may be utilized to extract a fluid and, in certain embodiments, release the fluid.

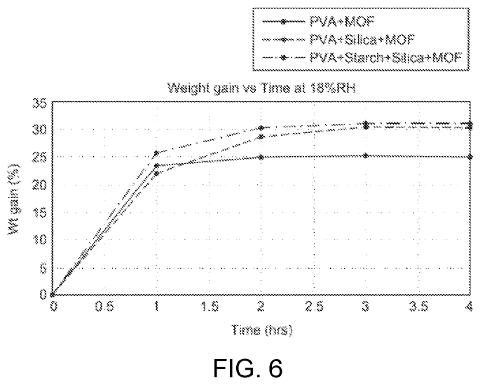

As described herein, the fluid capture material 44 may include a cross-linking agent 37 (i.e., used to cross-link the polymer forming the fluid capture material 44). In some embodiments, the cross-linking agent 37 may include colloidal silica. FIG. 6 shows a graph having an x-axis corresponding to time and a y-axis corresponding to weight gain (%). In the graph, the weight gain versus time is shown of gas capturing coating formed of PVA as a binder and MOF as a sorbent (i.e., ‘PVA+MOF’); PVA as a binder, and silica as a cross-linking agent, and MOF as a sorbent (i.e., ‘PVA+silica+MOF’); and silica and starch as a cross-linking agent, and MOF as a sorbent (i.e., ‘PVA+silica+starch+MOF’). As shown, the fluid capture material with a cross-linked agent (i.e., thereby having a cross-linked polymer composite matrix) has a relatively higher weight gain, corresponding to more target fluid 18 adsorbed to the fluid capture material 44.

### Enhanced Porosity.

The porosity of the films may be controlled and enhanced with the use of a pore precursor. For example, wax particles may be formulated into a MOF slurry formulation that is subsequently cast into a film. These wax particles take up space in the film, which can then be transformed to channels or pores once the wax particles are removed. The wax particles may be removed by any suitable means, such as heating to melt them and/or by washing the film with an appropriate solvent, and/or by performing a solvent extraction with an appropriate solvent. Heating may melt wax, remove certain materials by sublimation, and/or decompose compounds capable of generating gas. Once the wax particles are removed, the film has added porosity, which improves macropore diffusion.

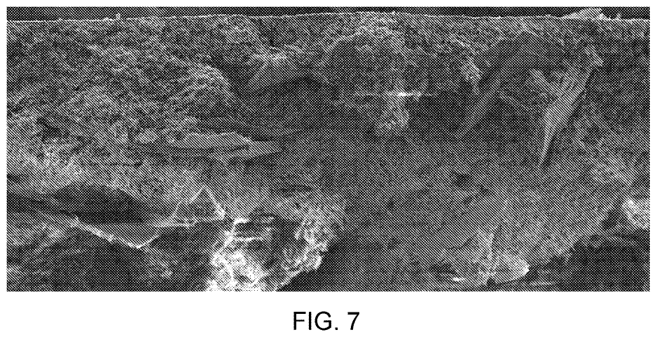

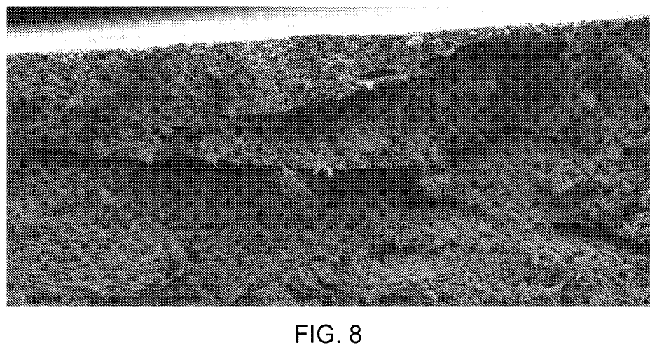

FIG. 7 depicts an SEM image of a cross-section of a film after casting and including wax (dicetyl fumarate) crystals. FIG. 8 depicts an SEM image of a cross-section of a film after casting and produced using wax crystals, where the wax crystals have been removed after heating to 90° C. Together, these figures demonstrate that a pore precursor, such as a wax crystal, may be included in a cast film and then subsequently removed to leave at least one pore.

### Methods of Preparing Films.

Film without Pores.

First 0.025 g of a polyvinyl butyral resin (Butvar B98) was dissolved in 1.8 g ethanol, then 0.025 g of clay was added to this solution and the result was stirred for 15 minutes. 0.5 g of amine-functionalized MOF sorbent was later added, and the slurry was vortexed for few minutes before coating it on 2″ by 2″ aluminum coupon. The coated coupon was air dried in the fume hood for an hour, then dried in the oven at 90° C. for an hour. The density of the film was measured at 0.26 g/cm³.

Film with Pores.

First 0.025 g of a polyvinyl butyral resin (Butvar B98) was dissolved in 1.8 g ethanol, then 0.025 g of clay was added to this solution and the result was stirred for 15 minutes. 0.1 g of wax (dimethyl eicosanedioate) was added to the solution and mixed for an hour. 0.5 g of amine-functionalized MOF sorbent added, and the slurry vortexed for few minutes before coating it on 2″ by 2″ aluminum coupon. The coated coupon was airdried in the fume hood for an hour, then submerged in heptane for 2 hours to remove the wax. The coupon was later removed from heptane, air dried, then dried in the oven at 90° C. for an hour. ¹H NMR was carried out to confirm the total removal of dimethyl eicosanedioate from the film. The density of the film was measured at 0.24 g/cm.

The kinetics of CO₂ adsorption of the film made with wax were 7 min faster than that of the film without pores.

### SUMMARY

Accordingly, the present disclosure relates to a fluid capture material or fluid capture material that provides improved fluid binding capacity and stability. The fluid capture material or coating generally includes a sorbent material and a binder material. The fluid capture material or coating also includes at least one pore formed by a pore precursor. As described herein, the resulting fluid capture material or coating may include a cross-linked polymer formed of one or more binder materials and certain cross-linking agents such as UV light, silica, polyacrylic acid, heat, or a combination thereof.

Technical effects of the invention include, and are not limit to, improving the capacity and/or capture efficiency of a substrate via a fluid capture material. By providing the disclosed fluid capture material, the amount of certain gases that remain in an exhaust gas flow may be facilitated to be reduced. Moreover, by forming a fluid capture material that includes a cross-linked polymer, a relatively higher amount of sorbent material compared to binder material may be used, thereby improving the fluid binding capacity of the fluid capture material. Finally, by forming a fluid capture material that includes at least one pore, the porosity and gas diffusion capabilities of the fluid capture material are enhanced.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A system, comprising:
a substrate; and
a fluid capture material formed on one or more surfaces of the substrate, wherein the fluid capture material comprises:
a sorbent material configured to bind one or more fluids comprising water, carbon dioxide, sulfur oxides, alcohols, or a combination thereof; and
one or more binder materials, wherein the binder material is optionally at least partially cross-linked;
wherein the fluid capture material comprises at least one pore.

2. The system of claim 1, wherein the fluid capture material comprises less than 15% by weight of the one or more binder materials.

3. The system of claim 1, wherein the sorbent material comprises a metal-organic framework (MOF), a covalent-organic framework (COFs), polymeric resins, silicas, zeolites, or a combination thereof.

4. The system of claim 1, comprising a cross-linking agent, wherein the binder material is at least partially cross-linked with the crosslinking agent, and wherein the cross-linking agent comprises one or more of a methacrylate reagent, an acrylate reagent, a vinyl ketone reagent, a vinyl reagent, or an allyl reagent.

5. The system of claim 1, comprising a cross-linking agent, wherein the binder material is at least partially cross-linked with the crosslinking agent, and wherein the cross-linking agent comprises polyacrylic acid.

6. The system of claim 1, wherein the one or more binder materials comprise vinyl polymers, starch, alkyl cellulose, or a combination thereof.

7. The system of claim 1, comprising a cross-linking agent, wherein the binder material is at least partially cross-linked with the crosslinking agent, and wherein a ratio of the cross-linking agent to the binder material is less than 25%.

8. The system of claim 1, wherein a thickness of the fluid capture material is between 0.1 and 3.5 mm.

9. The system of claim 1, wherein the fluid capture material comprises greater than 80% by weight of the sorbent material.

10. The system of claim 1, where the binder material that is at least partially cross-linked comprises a cross-linked density that is greater than 10%.

11. A method, comprising:
providing a sorbent material configured to bind one or more fluids comprising water, carbon dioxide, sulfur oxides, alcohols, or a combination thereof;
providing one or more binder materials, wherein the one or more binder materials optionally comprises a component capable of forming a cross-linked polymer;
optionally providing a cross-linking agent;
providing a pore precursor;
generating a sorbent-binder material based on the sorbent material, the one or more binder materials, optionally the cross-linking agent, and the pore precursor;
applying the sorbent-binder material to a substrate;
forming a fluid capture material using the sorbent-binder material applied to the substrate; and
removing the pore precursor to form at least one pore in the fluid capture material.

12. The method of claim 11, wherein forming the fluid capturing material comprises:
forming a first layer of the fluid capture material using the sorbent-binder material;
pre-wetting the first layer; and
forming a second layer onto the pre-wet first layer.

13. The method of claim 12, wherein the one or more binder materials comprise a first binder material and a second binder material, wherein the first binder material is distinct from the second binder material

14. The method of claim 11, wherein providing one or more binder materials comprises providing a first amount of the one or more binder materials, wherein providing the cross-linking agent comprises providing a second amount of the cross-linking agent, and wherein the ratio of the second amount to the first amount is less than 1/3.

15. The method of claim 11, wherein providing one or more binder materials comprises providing a first amount of the one or more binder materials, wherein providing the cross-linking agent comprises providing a second amount of the cross-linking agent, and wherein the ratio of the second amount to the first amount is less than 1/4.

16. A system, comprising:
a fluid capture material configured to bind one or more fluids, wherein the fluid capture material comprises:
a sorbent material configured to bind one or more fluids comprising water, carbon dioxide, sulfur oxides, alcohols, or a combination thereof; and
one or more binder materials:
wherein the fluid capture material comprises at least one pore;
wherein the one or more binder materials are optionally at least partially cross-linked; and
a fluid contactor having one or more surfaces coated with the fluid capture material.

17. The system of claim 16, wherein a thickness of the fluid capture material is between 0.1 mm and 2.0 mm on at least one surface of the one or more surfaces.

18. The system of claim 16, wherein a thickness of the fluid capture material is between 0.5 mm and 1.5 mm on at least one surface of the one or more surfaces.

19. The system of claim 16, wherein the fluid capture material comprises greater than 80% by weight of the sorbent material.

20. The system of claim 16, where the binder material that is at least partially cross-linked comprises a cross-linked density that is greater than 50%.
